(19) United States
(12) Patent Application Publication
(10) Pub. No.: US 2016/0193086 A1
(43) Pub. Date: Jul. 7, 2016

(54) BANDAGES WITH SYMBOLS AND METHODS OF FORMING SUCH BANDAGES

(71) Applicant: Frank Castle, New York, NY (US)

(72) Inventor: Frank Castle, New York, NY (US)

(21) Appl. No.: 14/989,998

(22) Filed: Jan. 7, 2016

Related U.S. Application Data
(60) Provisional application No. 62/100,589, filed on Jan. 7, 2015.

(51) Int. Cl. A61F 13/00

(52) U.S. Cl. CPC A61F 13/00059; A61F 13/00987; A61F 13/00008

(57) ABSTRACT

Bandages for covering a wound of a patient include a gauze material and a symbol applied to the gauze material. In some embodiments, bandages for covering a wound of a patient may include a gauze material and at least one code symbol applied to the gauze material. The at least one code symbol is selected from the group consisting of a numerical code, a bar code, a Quick Response code, and a color code, the at least one code symbol being associated with information selected from the group consisting of a warning, an instruction for use of the bandage, information about the wound, information about the bandage, and information about the patient. Methods of forming a bandage for covering a wound of a patient include directly applying a symbol to a gauze material.

### CROSS-REFERENCE TO RELATED APPLICATION

This application claims the benefit of U.S. Provisional Patent Application Ser. No. 62/100,589, filed Jan. 7, 2015, the disclosure of which is hereby incorporated herein in its entirety by this reference.

### FIELD

Embodiments of this application relate to medical devices generally and to, e.g., bandages for application to a human or animal body, such as to cover, support, or bind a bodily injury or wound, and to methods of forming such bandages.

### BACKGROUND

Many bandages for treating injuries or wounds are hypoallergenic, biocompatible, and sterile to avoid causing allergic reactions or infections. For example, gauze bandages may be directly applied to a wound and in direct contact with bodily fluids. Bandages are often white or another solid color with little, if any, decoration or symbols thereon. Some decorative bandages are commercially available, such as adhesive bandages marketed toward children. Such adhesive bandages may include cartoons, designs, or other decorations on an outer surface thereof.

### BRIEF SUMMARY

In some embodiments, bandages for covering a wound of a patient include a gauze material and a symbol applied to the gauze material.

In some embodiments, bandages for covering a wound of a patient include a gauze material and at least one code symbol applied to the gauze material. The at least one code symbol is selected from the group consisting of a numerical code, a bar code, a Quick Response code, and a color code, the at least one code symbol being associated with information selected from the group consisting of a warning, an instruction for use of the bandage, information about the wound, information about the bandage, and information about the patient.

In some embodiments, methods of forming a bandage for covering a wound of a patient include directly applying a symbol to a gauze material.

### BRIEF DESCRIPTION OF THE DRAWINGS

FIG. 1 shows a plan view of a bandage with a decorative symbol according to an embodiment hereof.

FIG. 2 shows a plan view of a bandage with a functional symbol according to an embodiment hereof.

### DETAILED DESCRIPTION

The following description provides specific details, such as material types and methodology, in order to provide a thorough description of embodiments of the disclosure. However, a person of ordinary skill in the art will understand that the embodiments of the disclosure may be practiced without employing these specific details. Indeed, the embodiments of the disclosure may be practiced in conjunction with, e.g., conventional fabrication techniques and materials employed in the industry.

In the following detailed description, reference is made to the accompanying drawings, which form a part hereof, and in which is shown, by way of illustration, specific embodiments that may be practiced. These embodiments are described in sufficient detail to enable a person of ordinary skill in the art to practice the disclosure. However, other embodiments may be utilized, and material, symbolic, and other changes may be made without departing from the scope of the disclosure. The illustrations presented herein are not meant to be actual views of any particular system, device, structure, or process, but are idealized representations which are employed to describe the embodiments of the disclosure. The drawings presented herein are not necessarily drawn to scale.

As used herein, the term “patient” means a human or other animal to which a bandage is applied.

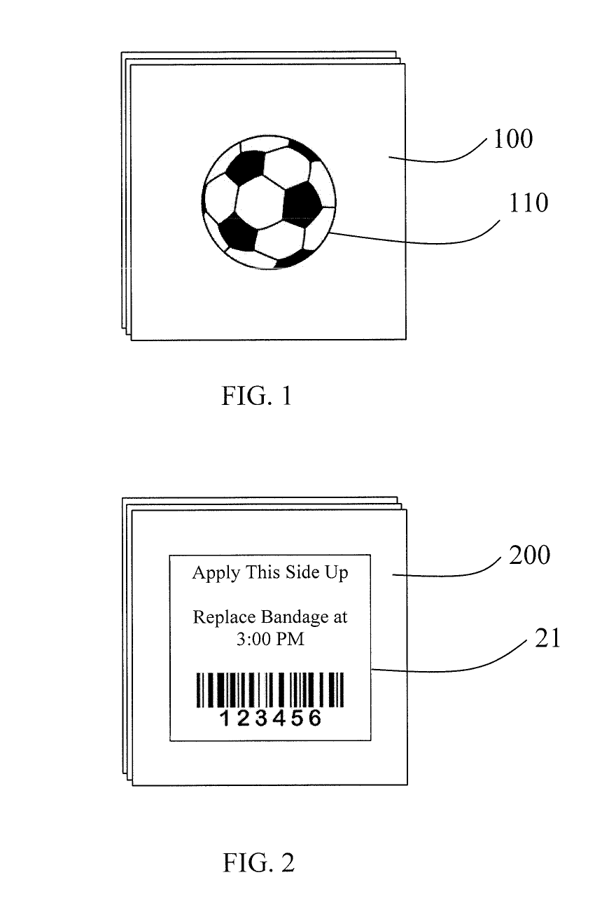

Referring to FIG. 1, a bandage 100 may include a symbol 110 thereon. The bandage 100 may be any bandage used for covering, dressing, supporting, or binding bodily injuries or wounds of patients. By way of non-limiting example, the bandage 100 may be a gauze bandage, such as a gauze pad, roller gauze (e.g., a gauze head wrap, a gauze bandage roll, a gauze strip commercially available under the trade name KERLIX®), a gauze sleeve bandage, a stretch gauze bandage, veterinary gauze, or an Unna boot wrapping. By way of other non-limiting examples, the bandage 100 may be a waterproof transparent dressing (e.g., a dressing commercially available under the trade name TEGADERM®), a triangular bandage, a pressure bandage, an elastic bandage wrap (e.g., an elastic bandage commercially available under the trade name ACE® or TENSOR®), a self-adhering wrap bandage (e.g., a bandage commercially available under the trade name CUBAN®), a cast wrap, moleskin, or a non-adherent dressing (e.g., a dressing commercially available under the trade name TELFA®).

The bandage 100 is shown in FIG. 1 generally as a multi-layer pad (e.g., a gauze pad) in a generally square shape. However, the present disclosure is not limited to a particular shape or a particular number of layers. Thus, the bandage 100 may be any shape (e.g., rectangular, circular, triangular, trapezoidal, irregular) and may have any number of layers (e.g., one, two, or more than two). The material of the bandage 100 may be woven or non-woven.

As shown in FIG. 1, the symbol 110 may be a decorative symbol 110, and may be any image, design, pattern, or representation. The decorative symbol 110 may be selected to cater to the interests of the patient, a family member or friend of the patient, or, if the patient is an animal, the patient's owner. By way of example and not limitation, the decorative symbol 110 may be an image of sports equipment (such as the soccer ball shown in FIG. 1), a sport's team logo, a cartoon character, a skull and crossbones, a flag, a sun, a moon, a star, an astrological symbol, a company logo, a happy face, an animal, a butterfly, a plant, a wave, a heart shape, a rainbow, a music symbol, a musical instrument, a spaceship, an automobile, an airplane, a bicycle, a robot, an anchor, a spiral, a Celtic knot, a playing card suit symbol, a yin-yang symbol, a religious symbol, a scriptural verse, a humorous saying, or an inspirational saying.

Since the bandage 100 with the decorative symbol 110 may be used on a patient's body and, in some cases, over an open wound, material of the decorative symbol 110 may be selected to reduce or avoid allergens, contaminants, and irritants. For example, the bandage 100 and decorative symbol 110 may be selected to comply with guidelines of the United States Food and Drug Administration. By way of further example, the material (e.g., dye or ink, adhesive, substrate, coating, thread) of the decorative symbol 110 may be one or more of hypoallergenic, sterile, resistant to deterioration, insoluble to fluids with which the bandage 100 may come in contact (e.g., water, saline solution, iodine, sweat, blood, other bodily fluids, rubbing alcohol), latex-free, irritant-free, non-toxic, and breathable. In some embodiments, the decorative symbol 110 may include one or more materials conventionally used in temporary tattoo decals, such as a transfer film (e.g., an adhesive gelatin or other polymeric material) and a dye or ink (e.g., glow-in-the-dark ink) printed on the transfer film. In some embodiments, the decorative symbol 110 may be formed of thread (e.g., dyed thread).

In embodiments in which the bandage 100 is a pad that is, for example, square, rectangular, or circular, the decorative symbol 110 may be located generally centrally on an outer surface (i.e., the surface to be placed away from the body of the patient) of the bandage 100. In other embodiments, the decorative symbol 110 may be located on the outer surface of the bandage 100 in any location that is visible and not covered by other portions of the bandage 100 when the bandage 100 is in normal use. By way of non-limiting example, if the bandage 100 is in the form of an elongated wrap or strip of material, the decorative symbol 110 may be located proximate (e.g., within about 12 inches of) a longitudinal end of the bandage 100 such that, when the bandage 100 is wrapped around a body part of the patient, the decorative symbol 110 may be positioned over other portions of the bandage 110 and, therefore, may be visible to the patient or to an external observer.

Although FIG. 1 illustrates a single decorative symbol 110 on the bandage 100, in some embodiments the bandage 100 may include multiple decorative symbols 110. For example, in embodiments in which the bandage 100 is an elongated wrap or strip of material, decorative symbols 110 may be located proximate each longitudinal end of the bandage 100 and/or on opposing flat surfaces of the bandage 100. Accordingly, at least one of the multiple decorative symbols 110 may be visible after application of the bandage 100 on a patient, regardless of which end of the bandage 100 is placed on the patient's body and covered by wrapping other portions of the bandage 100, and/or regardless of which flat surface of the bandage 100 is placed against the patient's body as the bandage 100 is in use. In some embodiments, the decorative symbol 110 may be repeated along a length of the bandage 100.

FIG. 2 illustrates another embodiment of a bandage 200 with a symbol 210 thereon. The bandage 200 may be essentially the same as the bandage 100 of FIG. 1 described above, except the symbol 210 may be a functional symbol 210 rather than the decorative symbol 110. The functional symbol 210 may include words, colors, numbers, codes (e.g., numerical codes, bar codes, Quick Response codes (“QR codes”), color codes), or other indicia to provide useful information to a patient and/or a caregiver. For example, the functional symbol 210 may include one or more of the following: an instruction for use (e.g., “Apply This Side Up,” “Replace Bandage at 3:00 PM” or another time), a warning (e.g., “Not for Internal Use”), information about the patient (e.g., allergies, name, patient tracking number), information about the wound being treated (e.g., “Open Wound,” date of injury, date of surgery), and information about the bandage 200 (e.g., “Latex-free,” “Includes Latex,” “Non-Adherent Bandage,” “Self-Adhesive Bandage,” “Includes Hydrocortisone” or another medicament). In embodiments in which the functional symbol 210 includes a code, the code may be used to track information about the patient and/or the wound being treated. For example, inputting the code into a computer system by, for example, entering a number or using a code scanner, may provide information to a caregiver to assist in treating the patient. For example, the code may be linked with information about the patient (e.g., allergies, name, age, health history), about the wound (e.g., date of injury or surgery, past evaluation information and times), about other caregivers (e.g., name of primary medical professional, name of nurses), and/or about treatment (e.g., times and dosage information for drugs, procedures for wound care).

Methods of forming the bandages 100, 200 may be the same regardless of whether the symbol 110, 210 is decorative or functional. Accordingly, manufacturing methods are described herein with reference to the bandage 100 of FIG. 1 as an example for the sake of convenience, but the description is relevant to the bandage 200 of FIG. 2 or another embodiment of a bandage in accordance with the present disclosure.

Referring again to FIG. 1, the bandage 100 with the symbol 110 thereon may be formed by different methods depending on the material and type of the symbol 110 and depending on the material and type of the bandage 100. For example, if the material of the symbol 110 is the same or similar to materials used for temporary tattoo decals, the symbol 110 may be printed on transfer film (e.g., an adhesive gelatin or other polymeric material) over a substrate film (e.g., a paper, plastic, or combined paper and plastic film), such as by screen printing, stamping, laser printing, or inkjet printing. The transfer film and the substrate film may be separated by a release coating, such as a silicone coating. The symbol 110 and the transfer film may be transferred to the bandage 100 and the substrate film removed. For example, the substrate film may be peeled and/or dissolved away from the transfer film after application of the symbol 110 and transfer film to the bandage 100.

By way of another non-limiting example, if the material of the symbol 110 is a thread (e.g., dyed thread), the symbol 110 may be directly embroidered onto the bandage 100. Alternatively, the symbol 110 of thread may be separately formed into a patch, which may be adhered or sewn onto the bandage 100.

The embodiments of the disclosure described above and illustrated in the accompanying drawing figures do not limit the scope of the invention, since these embodiments are merely examples of embodiments of the disclosure. The invention is encompassed by the appended claims and their legal equivalents. Any equivalent embodiments lie within the scope of this disclosure. Indeed, various modifications of the present disclosure, in addition to those shown and described herein, such as other combinations and modifications of the elements described, will become apparent to those of ordinary skill in the art from the description. Such embodiments, combinations, and modifications also fall within the scope of the appended claims and their legal equivalents.

What is claimed is:

1. A bandage for covering a wound of a patient, the bandage comprising:
a gauze material; and
a symbol applied to the gauze material.

2. The bandage of claim 1, wherein the symbol is a decorative symbol.

3. The bandage of claim 1, wherein the symbol is a functional symbol.

4. The bandage of claim 3, wherein the functional symbol provides information selected from the group consisting of a warning, an instruction for use of the bandage, information about the wound, information about the bandage, and information about the patient.

5. The bandage of claim 3, wherein the functional symbol is a code.

6. The bandage of claim 5, wherein the code comprises at least one of a numerical code, a bar code, a Quick Response code, and a color code.

7. The bandage of claim 1, wherein a material of the symbol comprises an adhesive transfer film and a dye or ink.

8. The bandage of claim 1, wherein a material of the symbol comprises thread.

9. The bandage of claim 8, wherein the thread is embroidered directly on the gauze material.

10. The bandage of claim 8, wherein the symbol comprises a patch sewn or adhered onto the gauze material.

11. A bandage for covering a wound of a patient, the bandage comprising:
a gauze material; and
at least one code symbol applied to the gauze material, the at least one code symbol selected from the group consisting of a numerical code, a bar code, a Quick Response code, and a color code, the at least one code symbol being associated with information selected from the group consisting of a warning, an instruction for use of the bandage, information about the wound, information about the bandage, and information about the patient.

12. A method of forming a bandage for covering a wound of a patient, the method comprising:
directly applying a symbol to a gauze material.

13. The method of claim 12, wherein directly applying a symbol to a gauze material comprises directly applying a decorative symbol to the gauze material.

14. The method of claim 12, wherein directly applying a symbol to a gauze material comprises directly applying a functional symbol to the gauze material.

15. The method of claim 11, wherein directly applying a symbol to a gauze material comprises directly applying the symbol to a gauze pad.

16. The method of claim 15, wherein directly applying the symbol to a gauze pad comprises generally centrally locating the symbol on an outer surface of the gauze pad.

17. The method of claim 12, wherein directly applying a symbol to a gauze material comprises directly applying the symbol to an elongated gauze strip.

18. The method of claim 17, wherein directly applying the symbol to an elongated gauze strip comprises locating the symbol within about 12 inches of a longitudinal end of the elongated gauze strip.

19. The method of claim 12, wherein directly applying a symbol to a gauze material comprises:
printing the symbol on a transfer film over a substrate film;
positioning the symbol and transfer film on the gauze material; and
removing the substrate film from the transfer film.

20. The method of claim 12, wherein directly applying a symbol to a gauze material comprises directly embroidering the symbol from thread on the gauze material.

21. The method of claim 12, wherein directly applying a symbol to a gauze material comprises:
forming the symbol from thread to form a patch; and
applying the patch to the gauze material by sewing or adhering the patch to the gauze material.
